(19) United States
(12) Patent Application Publication
(10) Pub. No.: US 2015/0088106 A1
(43) Pub. Date: Mar. 26, 2015

(54) METHOD FOR THE RENEWAL OF BIOLOGICAL TISSUES AND DEVICE FOR THE IMPLEMENTATION THEREOF (EMBODIMENTS)

(71) Applicants: Uladzimir Valiantinavich Khomchanka, Minsk (BY); Dmitry Vladislavovich Gorbach, Minsk (BY); Aliaksandr Valerjavich Sukhadolau, Minsk (BY)

(72) Inventors: Uladzimir Valiantinavich Khomchanka, Minsk (BY); Dmitry Vladislavovich Gorbach, Minsk (BY); Aliaksandr Valerjavich Sukhadolau, Minsk (BY)

(21) Appl. No.: 14/400,338

(22) PCT Filed: Sep. 6, 2012
(86) PCT No.: PCT/BY2012/000002

§ 371 Date: Nov. 19, 2014

(30) Foreign Application Priority Data
May 11, 2012 (EA) 201200845

(51) Int. Cl. A61B 18/20; A61N 7/00

(52) U.S. Cl. CPC A61B 18/203; A61N 7/00; A61B 2018/0047
USPC 606/9

(57) ABSTRACT

The invention relates to medicine and can be used in surgery, including cosmetic surgery, for example, for treating trophic and slowly healing ulcers, bed sores, burns, scars, etc., as well as for biological tissue rejuvenation, including skin, in different locations. The proposed method is for the regeneration of biological tissues with restoration of functional properties, characteristics and structure thereof, in which tissues are subjected to a predetermined degree of mechanically-induced trauma through the creation, in the desired areas, of at least one region of interference between acoustic waves generated by at least two sources and propagating in the tissues to be regenerated, with the possibility of subsequent natural regeneration of the corresponding biological tissues in said areas. Also proposed are various embodiments of the device for the implementation of the above method. To achieve the rejuvenation effect of different biological tissues located at different depths, the microtrauma areas are created with no thermal effect, i.e. neither evaporation nor coagulation of all overlying tissues, i.e. regeneration of the tissues occurs with no fibrous cell growth, suggesting that not only visual but also actual rejuvenation took place.

The invention relates to medicine and can be used in surgery, including cosmetic surgery, for example, for treating trophic and slowly healing ulcers, bed sores, burns, scars, etc., as well as for tissue rejuvenation, including skin, in different locations. The method is based on the transmission of non-mechanical energy, in particular, acoustic wave energy, into the human tissue. The invention also relates to different embodiments of the device generating the wave energy necessary for implementing said method.

Modern surgery, including cosmetic surgery, widely uses laser energy, ultrasound energy and similar non-mechanical types of energy for treatment and rejuvenation. Thus there is a known skin rejuvenation method comprising ablation or vaporization of superficial skin layers with carbon dioxide laser radiation at 10.6 μm wavelength or with Er laser (EnYAG) at 2.94 μm wavelength (Palomar 2940 Fractional Laser, Deka SmartXide DOT, Candela C02RE) [1]. The therapeutic effect in this case is based on the vaporization of superficial skin layers with minor thermal damage of deep dermic layers, which does not fully destruct all skin layers but stimulates new cell growth. Additional disadvantages of said method are the high trauma rate, long recovery, and wound formation, which carries a high risk of infection, pain, both during the procedure and during recovery, the risk of altered skin pigmentation and scar formation. In addition, the method cannot be used on moving body parts, such as neck, eyelids, etc., since wound healing requires immobilization of the treatment zone.

A method for non-invasive photorejuvenation, wherein radiation penetrates deeper into the skin, causing trauma to collagen fibers and then stimulating new collagen synthesis, is known in the art (Palomar 1540 Fractional Laser, Candela GentleMAX, Candela Smoothbeam) [2]. Said method can be used in the treatment of essentially all skin areas. The disadvantages of said method include inefficient therapeutic and aesthetic effects. The amount of synthesized collagen is insufficient for producing the rejuvenation effect expressed as diminished wrinkle size. The method improves skin color by increasing capillary blood supply and can cause temporary cutaneous edema, which creates a temporary wrinkle-reducing effect.

Methods for non-invasive ultrasonic skin rejuvenation are also known in the art [3,4,5]. Said methods are used for the treatment of various skin areas in the desired treatment sites. The disadvantages of said methods include the ultrasonic wave front effect on the tissue, which not only impacts the desired site but also the surrounding tissue, which, in turn, enlarges the trauma area and delays recovery.

The method most closely related to the claimed method is the microablative skin photorejuvination [6]. In said method, rather than treating the skin surface with one wide laser beam, the skin surface is treated with a plurality of microbeams. Each microbeam triggers either coagulation alone, coagulation combined with evaporation, or cutaneous microdomain ablation, depending on spectral and temporal parameters of the applied radiation. Microbeam diameters can span from 1 μm to hundreds micrometers, and they can be situated hundreds of micrometers apart. The therapeutic effect of the method is based on the assumption that the removed or damaged tissue will be replaced with new skin cells, and old skin will be completely replaced with new skin in the treatment area over the course of several sessions. Skin microcoagulation with Erbium glass lasers (laser apparatus Fraxel, wavelength 1.54 μm) induces thermal destruction of skin cells without evaporation thereof. Radiation causing both skin cell evaporation and coagulation (such as carbon dioxide radiation, wavelength 10.64 μm) produces microchannels of the evaporated skin surrounded by a coagulation zone. Erbium radiation laser (wavelength 2.94 μm) causes tissue evaporation as microchannels without coagulation of the surrounding tissue. Disadvantages of the method are include:
The depth of microtrauma, wherein the rejuvenation effect occurs, is limited by the coagulation or ablation depth, which does not allow for rejuvenation in the deep dermis or hypodermis;
The small depth of microtrauma is not useful for the enhanced tissue regeneration when treating trophic or septic wounds, etc., i.e. when tissue regeneration must occur at a much deeper level;
The method is invasive, which increases the risk of infection in the treated surface;
When tissue is removed, the living tissue is exposed to the environment, which can promote the growth of fibrous tissue instead of full-fledged rejuvenation of the unaltered tissue;
Microablative procedures are painful and thus require the use of anesthetics;

Thus, the object of the present invention is to provide a noninvasive method for the rejuvenation of biological tissue and restoration of functional properties, characteristics, and structure thereof by creating microtrauma sites in the desired areas of biological tissue, causing natural regeneration of the corresponding biological tissue in the desired areas. Microtrauma (microdestruction) inside the biological tissue should not lead to the formation of microchannels exposed to the aggressive environment, which would completely preclude the formation of fibrous tissue. The treatment should promote the formation of microtrauma sites and later regeneration of both the superficial and deep biological tissue of any localization and any type. The method should also reduce pain and the risk of infection as compared to other methods known in the art, including microablattive methods

The stated objective is achieved in the claimed method for the rejuvenation of biological tissue and restoration of functional properties, characteristics, and structure thereof by creating microtrauma sites in the desired areas of biological tissue, followed by natural regeneration of the corresponding biological tissue in the desired areas with specified mechanically-induced trauma by creating in the desired areas at least one site of acoustic interference generated by at least two sources and propagating in the tissue to be regenerated.

In the claimed method, the powerful common-mode acoustic waves with the desired (calculated) characteristics, such as power, are generated, in general, on the surface to be rejuvenated or the surface of overlying biological tissue. In particular, altering the power of acoustic waves can have a corresponding effect on the predetermined depth of microtrauma areas. The interference effect of interacting waves in the method of the present invention decreases the size and determines the exact location in all directions of the tissue area subjected to microtrauma, which results in a considerably higher efficiency of the directional energy effect and shorter recovery time. In the claimed method, the tissue trauma is nonthermal and thus, the effect thereof on the biological tissue does not cause evaporation or coagulation. Additionally, since acoustic waves can penetrate the biological tissue at a set depth (defined by the acoustic wave characteristics) with no channel formation, the effect of the present invention does not increase the contact between the living tissue and oxygen, which impedes the growth of fibrous tissue and promotes real and not only visual regeneration/rejuvenation of biological tissue. Energy of the acoustic waves, which is stronger in the desired interference zones, does not destruct tissue but causes trauma to the selected tissue areas. Because regeneration of biological tissue can occur not only as a result of complete tissue cell destruction, but also as a result of partial trauma thereof, the biological tissue in the selected locations are regenerated. Since no complete destruction of deeply underlying cells of biological tissue takes place, recovery time is greatly reduced. Since in the method of the present invention, the trauma area is reduced, the level of trauma can be preset, and the thermal effect is absent, the pain during the procedure is considerably reduced.

The areas of the common-mode acoustic wave sources are preferably 10 nm² to 10 μm². Preferably, all epicenters of acoustic waves are located at equal distances from one another, selected from the 10 μm to 1 cm range.

According to the present invention, mechanical trauma zones are preferably formed below the surface of tissue exposed to the environment, without increasing the contact surface of living tissue with the aggressive media. Thus, the claimed method creates microtrauma areas with no expansion of contact areas between the living tissue and the environment, which considerably reduces the risk of infection during recovery in comparison to the methods known in the art.

The minimum power of the generated acoustic waves is selected in such a way that:
the power of a single wave generated by one epicenter would be insufficient to cause mechanical trauma/destruction of the treated biological tissue;
the combined power of the interference of the waves generated by adjacent epicenters would be sufficient to cause trauma of the desired level to the treated biological tissue.

Erythema that appears on the skin surface after treatment can serve as visual control of the sufficiency of the impact.

In some preferred embodiments of the present invention, the initial power of each single acoustic wave is selected in such a way that mechanically traumatized biological tissue areas are formed both in the interference zone and in the zone at least immediately surrounding the epicenter of said acoustic wave.

Another important characteristic of acoustic waves, which can be varied to affect the biological tissue, is frequency. Thus, the frequency of the acoustic waves corresponding to the inherent natural frequencies of any given biological tissue can be selected to synchronize with said frequencies to achieve their selective regenerative effect on specific biological tissues. Therefore, in several preferred embodiments of the present invention in accordance with the natural frequencies of the biological tissue to be rejuvenated, the frequency of the acoustic wave is selected to exert a selective effect only on the biological tissue to be rejuvenated.

In preferred embodiments of the present invention, the level of mechanical trauma is selected from the range starting from the level causing destruction of the cell membrane integrity and ending with the level causing full destruction of the cells in the tissue to be rejuvenated. Consequently, the effect of the present invention can stimulate regeneration of biological tissue with the destruction of entire cells of said tissue or with no destruction. The regeneration/rejuvenating effect can be achieved even with partial trauma to the biological tissue cells.

Other preferred embodiments include those where the acoustic waves are generated as directional acoustic waves, which also contribute to the localization and optimization of microtrauma site formation.

The set objective is also achieved in various embodiments of the claimed device for the implementation of the aforementioned claimed method for the rejuvenation of biological tissue and restoration of functional properties, characteristics, and structure thereof, comprising a source of radiation and a device for the formation of a plurality of acoustic wave epicenters on the superficial and overlying biological tissue to be rejuvenated.

In the first embodiment of the claimed device, the set objective is achieved with the radiation source being an ultrasound generator equipped with a device that can form a plurality of acoustic wave epicenters located at equal distances from one another on the superficial biological tissue to be rejuvenated or the overlying biological tissue.

In the second embodiment of the claimed device, the set objective is achieved with the radiation source being a laser source and the device, which can form a plurality of acoustic wave epicenters on the surface to be rejuvenated or the overlying biological tissue, being a substance capable of providing selective absorption of waves with a desired wavelength, which is applied to the surface of biological tissue at the desired pinpointed sites of preset sizes located at equal distances from one another.

The preferred distances between epicenters for the first and second embodiments of the claimed device are 10 μm to 1 cm.

In the third embodiment of the claimed device, the set objective is achieved with the radiation source being a laser light source, and the device, which can form a plurality of acoustic wave epicenters on the surface to be rejuvenated or the overlying biological tissue, being a substance capable of converting the spatial distribution of beam intensity to form a periodic structure on the surface of said biological tissue with maxima and minima of light energy. Said laser light source is configured to generate radiation with parameters that can provide efficient absorption thereof by biological tissue and produce acoustic waves at the points of maximum light energy.

The aforementioned and other benefits and advantages of the claimed method for the rejuvenation of biological tissue and restoration of functional properties, characteristics, and structure thereof, and the embodiments of the corresponding devices will be further disclosed in detail in the examples of some possible preferred but non-limiting embodiments with references to the accompanied drawings and figures in which:

FIG. 1: is a schematic of the formation of microtrauma areas in biological tissue cells in one (first) of the possible embodiments;

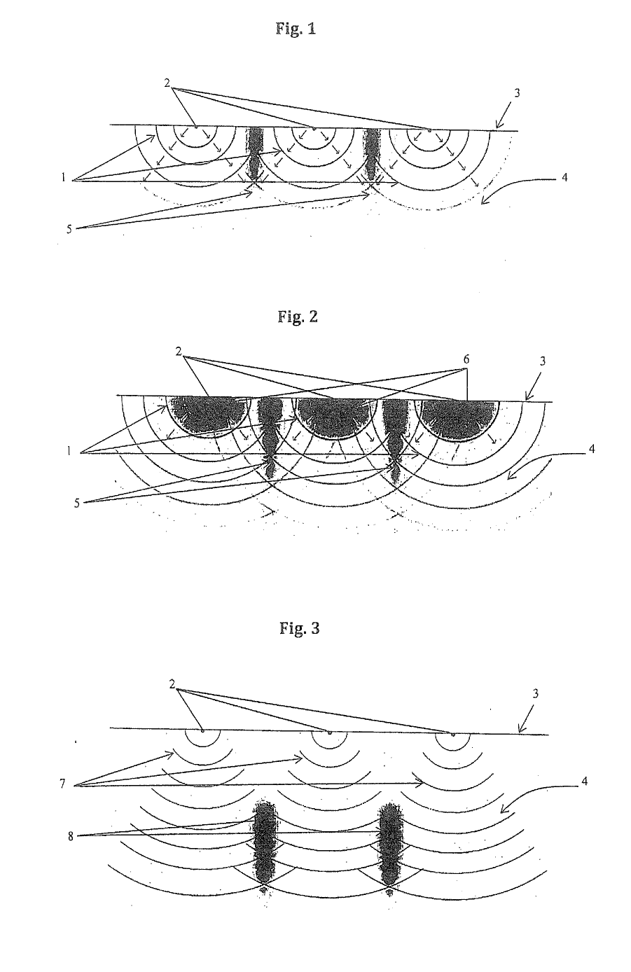

FIG. 2: is a schematic of the formation of microtrauma areas in biological tissue cells in another (second) possible embodiment;

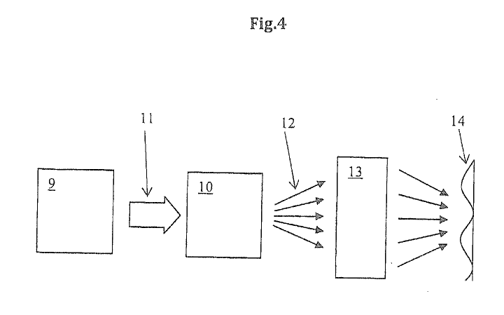

FIG. 3: is a schematic of the formation of microtrauma areas in biological tissue cells in yet another (third) possible embodiment;

FIG. 4: is a schematic diagram of the device in the third embodiment.

FIG. 1 shows a schematic of the formation of microtrauma areas in biological tissue cells in one of the possible embodiments, wherein acoustic waves 1 travel from corresponding epicenters 2 on surface 3 of biological tissue 4 into biological tissue 4, wherein the power of each acoustic wave 1 is selected in such a way that it is not sufficient for the mechanical destruction of any components of the treated biological tissue 4. Microtrauma areas will occur only in interference zones 5 (marked with dark shading on the drawing) of acoustic waves 1 generated by adjacent epicenters 2.

FIG. 2 shows a schematic of the formation of microtrauma areas in biological tissue cells in another (second) possible embodiment, wherein acoustic waves 1 travel from corresponding epicenters 2 on surface 3 of biological tissue 4 into biological tissue 4, wherein the power of each acoustic wave 1 is selected in such a way that it creates areas 6 of mechanical destruction of the treated biological tissue 4 near the corresponding epicenter 2 (full destruction of superficial tissue) and microtrauma areas (local trauma areas) in interference zones 5 of acoustic waves 1 generated by adjacent epicenters 2. Mechanical destruction zones 6 and interference zones 5 are marked with dark shading on the drawings.

FIG. 3 shows a schematic of the formation of microtrauma areas in biological tissue cells in yet another (third) possible embodiment, wherein acoustic waves 1 travel from corresponding epicenters 2 on surface 3 of biological tissue 4 into biological tissue 4, wherein in order to create microtrauma areas in deep tissue, acoustic waves are generated as directional acoustic waves 7. Microtrauma areas are formed in interference zones 8 of every two directional acoustic waves 7 generated by corresponding adjacent epicenters 2. Interference zones 8 are marked with dark shading on the drawings.

FIG. 4 shows a schematic diagram of the device in the third embodiment, wherein the radiation device is laser light source 9, and the device for the formation of a plurality of acoustic wave epicenters on the surface of biological tissue to be rejuvenated or overlying biological tissue comprises beam splitter 10 splitting initial laser beam 11 into plurality of beams 12 necessary to achieve the desired distribution, and optical system 13, which collimates beams 12, formed by beam splitter 10, at angles 14 required to achieve the desired spatial distribution of beam intensity by means of multibeam interference.

The claimed method is carried out as follows:

Using any of the embodiments of the claimed method for the rejuvenation of biological tissue and restoration of functional properties, characteristics, and structure thereof, acoustic waves 1 (7) are generated with the desired characteristics (power, frequency). Each acoustic wave 1 (7) starts traveling into biological tissue 4 from corresponding epicenter 2. The microtrauma areas are created following the procedure of a particular embodiment of the claimed method.

For instance, for the embodiment of FIG. 1, since the minimum power of acoustic wave 1 is insufficient for the mechanical destruction of any of the components of treated biological tissue 4, the wave propagation is not accompanied by the destruction of biological tissue 4. However, interference with acoustic waves 1 propagated from corresponding adjacent epicenters 2 generates a localized cumulative power of acoustic wave 1 at the levels sufficient for the creation of a certain degree of mechanical trauma areas on biological tissue 4 in said interference areas 5. In addition, said areas will penetrate deeply inwards from surface 3 and will becomes zones of uneven (of the desired level) microtrauma of biological tissue cells 4.

Such tissue trauma is not intended to enlarge the contact area of living tissue with the aggressive environment, which minimizes the fibrous tissue formation.

The power of acoustic waves 1 can be increased, according to the embodiment of FIG. 2, to reach the level when each separate acoustic wave 1 can independently destruct tissue. In that case, the effect will be produced in a somewhat different manner and will occur as follows: waves 1 propagating from each epicenter 2 into biological tissue 4 will trigger mechanical destruction thereof (areas 6 of mechanical destruction) until the power of said waves falls below the threshold. Any further propagation of waves 1 into biological tissue 4 is not accompanied by the destruction thereof, except interference zones 5 of waves 1 propagating from adjacent epicenters 2. Thus, the trauma zone will include a completely destructed superficial tissue area (zone 6 of mechanical destruction) and local trauma zones (zones 5 of interference).

The observed visual effect in this case is the appearance of “frost”, i.e. a mechanically destructed area, on the treated surface. Additionally, varying the power of the acoustic wave can vary the depth of microtrauma area locations.

Microtrauma to deep tissue 4 with subsequent regeneration thereof is conducted in accordance with the third embodiment (see FIG. 3) of the claimed method, i.e. by generating directional acoustic waves 7. In this case, the interference of acoustic waves 7 will take place only deep inside the biological tissue with no trauma to the superficial layers. Mechanical trauma areas will occur in interference zones 8.

Tissue regeneration in the embodiment of the present invention occurs faster compared to the prototype, because even in the effect of the second embodiment (see FIG. 2), the only fully mechanically destructed tissue is superficial tissue (area 6 of mechanical destruction), while deep tissue is not fully destructed but only traumatized (interference zones 5). In all other embodiments, including those not individually disclosed in the present specification, there is no full destruction of any biological tissue whatsoever.

As to the device for the implementation of the claimed method for the rejuvenation of biological tissue, there are various possible embodiments of said method.

Thus, in the device according to the first embodiment, the radiation source can be configured as a powerful ultrasound generator. The “former” of acoustic waves 1 of epicenter 2 (7) can be configured as a contact pad, which is a ranked set of needles with a preset step (at 10 μm to 1 cm distance from one another) and contacting with surface 3 of biological tissue 4 in the 10 nm² to 10 μm² areas.

In the device according to the second embodiment, the radiation source is configured as a powerful generator of intense pulsed light. The “former” of acoustic waves 1 of epicenter 2 (7) is configured as a special absorbing medium (substance) with high absorption constants for the selected emission wavelength, which is applied to the surface being treated. The absorbing medium must be applied as separate points with 10 nm² to 10 μm² areas, and 10 μm to 1 cm distances between one another.

In the device according to the third embodiment, the radiation source can be configured as a powerful generator of intense laser light, which is readily absorbed by the biological tissue to be treated. In the present instance, there is no need to apply any additional substance to surface to be treated 3, but the laser beam has to be laterally distributed in such a way that said distribution would form a periodic structure on the treated surface with maxima and minima of energy. In addition, high-energy areas must be in the 10 nm² to 10 μm² range (at 10 μm to 1 cm distances from one another). Thus, the device of the third embodiment is based on the multibeam interference phenomenon and is intended to include the transformation of the initial laser beam into the desired final spatial intensity distribution on the surface to be treated or the surface of the overlying biological tissue.

A schematic diagram of said device is shown on FIG. 4. Wide laser beam 11, generated by laser light source 9, falls on beam splitter 10, which divides said beam into plurality of beams 12, which are required to achieve the desired distribution. Optical system 13 collimates beams 12, formed by beam splitter 10, at angles 14 necessary to achieve the desired spatial intensity distribution by means of multibeam interference 14.

Beam splitter 10 is an optical element or device splitting initial laser beam 11 into plurality of beams 12 of the required intensity. Beam splitter 10 can be constructed based on the following groups of elements or combinations thereof:
phase or amplitude gratings, or more complex diffractive elements,
mirrors with dielectric or metallic coating, metal mirrors,
spherical and/or cylindrical lenses with or without dielectric or metallic coating,
prisms with or without dielectric or metallic coating
microlens and microprism systems with or without dielectric coating.

Optical system 13 is an is an optical element or device combining beams 12 generated by beam splitter 10 in the plane of resulting spatial intensity distribution 14. Said optical system delivers the beams onto the plane of resulting spatial intensity distribution 14 at angles necessary for reaching the desired resulting intensity distribution 14. Optical system 13 can be constructed based on the following groups of elements or combinations thereof:
spherical and/or cylindrical lenses with or without dielectric or metallic coating,
mirrors with dielectric or metallic coating, metal mirrors,
prisms with or without dielectric or metallic coating

Resulting spatial intensity distribution 14 is an ordered periodic system of interferential maxima and minima of beam intensities with preset sizes and frequencies on treated surface 3 or overlying biological tissue 4.

The device according to the third embodiment, shown on FIG. 4, functions in a wide wavelength range, 200 nm to 20 μm, and a wide range of characteristic maxima and minima sizes of spatial intensity distribution 14 of beam intensity: from the wavelength of the applied radiation to 1 mm.

Particularly, conversion of Er:YAG laser radiation, set at 2.94 μm wavelength, can be effected via beam splitter 10 comprising two phase gratings constructed from silica glass KI with a U-shaped-profile grating. The grating grooves are perpendicular to one other. The groove depth is selected to eliminate the zero diffraction order in the diffraction pattern. Thenceforth, four beams 12 of the first diffraction order receive 80% of the initial radiation energy (beam 11). Beams 12 are paired in mutually perpendicular planes. Diffraction orders that are above four beams 12 of the first order are filtered off with a special diaphragm.

Resulting beams 12 are collimated by optical system 13, comprising two lenses, into the plane of final spatial beam intensity distribution 14.

Spatial beam intensity distribution 14 is then created as alternating maxima and minima. Resulting intensity distribution 14 is sinusoidal with the 100-μm period along the lines aligned with the grating orientation of beam splitter 10.

The above description, illustrated with some possible non-limiting embodiments, therefore, demonstrates that although methods and corresponding devices for the regeneration/rejuvenation of biological tissue are known in medical practice, the claimed method and device provide novel and unexpected technical results. Said results are achieved primarily because for the rejuvenation of various biological tissue located at different depths, the areas of micotrauma can be created with no thermal effect applied, i.e., no evaporation or coagulation of any of the overlying tissue. Thus, tissue regeneration occurs with no fibrous cell growth, suggesting that not only visual but also actual rejuvenation takes place.

### REFERENCES

1. B. Eremeev, K. Kalaydzhyan. Lasers Against Wrinkles. Electronic almanac “Cosmetics & Medicine”. [Electronic resource]—Apr. 6, 2012. Access mode: http://daniel.ru/cm/arc/r403.htm.
2. Palomar-Rejuvenation. Website of the medical center RODEN. [Electronic resource]—May 4, 2012.—Access mode: http://www.roden.by/cosmetology/palomar-omolojenie/
3. Application US2011/0218464 A1, publ. Sep. 8, 2011.
4. Application US2012/0016239 A1, publ. Jan. 19, 2012.
5. Application US2012/0053458 A1, publ. Mar. 1, 2012.
6. U.S. Pat. No. 6,997,923 B2, publ. Oct. 31, 2002.

1. A method for the rejuvenation of biological tissue and restoration of functional properties, characteristics, and structure thereof by creating microtrauma sites in the desired areas of biological tissue, followed by natural regeneration of the corresponding biological tissue in specified areas, wherein said tissue are subjected to specified mechanically-induced trauma by affecting the desired areas with at least one site of acoustic interference generated by at least two sources and propagating through the tissue to be regenerated.

2. The method according to claim 1, wherein all epicenters of the acoustic waves are located at equal distances from one another, selected from the 10 μm to 1 cm range.

3. The method according to claim 1, wherein the mechanical trauma zones are formed below the surface of tissue exposed to the environment, without increasing the contact surface of living tissue with aggressive media.

4. The method according to claim 1, wherein the initial power of each single acoustic wave is selected in such a way that the mechanically traumatized biological tissue areas are formed both in the interference zone of said wave with at least one of the adjacent waves and in the zone at least immediately surrounding the epicenter of said acoustic wave.

5. The method according to claim 1, wherein the frequency of the acoustic wave is selected to correspond to the natural frequency of the desired biological tissue to provide a selective regenerative effect only on said biological tissue to be rejuvenated.

6. The method according to claim 1, wherein the level of mechanical trauma is selected from the range between the level providing destruction of cell membrane integrity only and the level providing full destruction of cells in the tissue to be rejuvenated.

7. The method according to claim 1, wherein the acoustic waves are generated as directional acoustic waves.

8. A device for the rejuvenation of biological tissue and restoration of functional properties, characteristics, and structure thereof of the present method according to claim 1, comprising a radiation source configured as an ultrasound generator equipped with a device forming a plurality of acoustic wave epicenters located at equal distances from one another on the superficial biological tissue to be rejuvenated or the overlying biological tissue.

9. The device according to claim 8, wherein the distance between acoustic wave epicenters is 10 μm to 1 cm.

10. The device for the rejuvenation of biological tissue and restoration of functional properties, characteristics, and structure thereof of the present method according to claim 1, comprising a laser light source and a device forming a plurality of acoustic wave epicenters configured as a substance capable of providing selective absorption of waves with desired wavelengths, which is applied to the surface of biological tissue at the desired pinpointed sites with preset sizes located at equal distances from one another.

11. The device according to claim 10, wherein the distance between acoustic wave epicenters is 10 μm to 1 cm.

12. The device for the rejuvenation of biological tissue and restoration of functional properties, characteristics, and structure thereof of the present method according to claim 1, comprising a laser light source and a device forming a plurality of acoustic wave epicenters configured as a substance capable of converting spatial distribution of beam intensity to form a periodic structure on the surface of said biological tissue with maxima and minima of light energy, wherein said laser light source is configured to generate radiation with parameters affording efficient absorption thereof by biological tissue and produce acoustic waves in the points of maximum light energy.
